(19) United States
(12) Patent Application Publication
(10) Pub. No.: US 2023/0255509 A1
(43) Pub. Date: Aug. 17, 2023

(54) SYSTEM AND METHOD FOR ACCURATE METABOLIC RATE CALCULATION

(71) Applicant: Hussam Zebian, Milpitas, CA (US)

(72) Inventor: Hussam Zebian, Milpitas, CA (US)

(21) Appl. No.: 18/306,218

(22) Filed: Apr. 24, 2023

Related U.S. Application Data
(63) Continuation-in-part of application No. 18/057,175, filed on Nov. 18, 2022.
(60) Provisional application No. 63/281,612, filed on Nov. 19, 2021.

(51) Int. Cl. A61B 5/083; A61B 5/0205

(52) U.S. Cl. CPC A61B 5/0833; A61B 5/0205

(57) ABSTRACT

The present disclosure is directed to a method and system of detecting and calculating a lung map or lung properties of an individual user from nonintrusive wearable sensors to generate a metabolic rate of the user as well as several other key body and performance metrics.

### BACKGROUND

### Technical Field

The present disclosure is directed to a system and method for more accurate calculation of metabolic rate of a user based on quantifying the boundary properties of the user's lung.

### Description of the Related Art

The current technology has limited success in calculating the metabolic rate of a user.

The wearable technology is a huge industry, and its size is increasing with the increased heath concerns of the population, increase in the data driven approach of users, and the general shift of all generations, in particular the newer generations, into everything tech. However, it is easy to see that what the wearable technology industry currently delivers are inaccurate estimations when it comes to the metabolic rate or calorie expenditure calculations. The current technology relies mainly on the heartrate measurement from the user. Some make corrections based on the number of steps taken (if the activity relates to taking steps) to calculate the resulting calorie expenditure. Numerous academic and research studies have shown that the results of calorie expenditure reported by the current technology are inaccurate, over 30% error compared to actual expenditure. It is noteworthy to elaborate that the sensors in these wearables measure the body signals very accurately (i.e. the heartrate and the steps taken count are very accurate) but the calculations and assumptions to get to metabolic rate and energy expenditure or consumption are not accurate.

This leads us to realize that it is not a matter of sensor technology error, instead it is an error in what they claim to be the theory and model behind their technology. Heartrate alone is inadequate for calculating the metabolic rate. The current technology of wearable relies on measuring the heartrate of user and plugging it into a model obtained by regressions from lab studies. In those lab studies, a number of test subjects perform physical activity, mainly a mono-structural movement with limited position changes to facilitate extracting data (like running on a treadmill or stationary cycling), while connected to a gas exchange analyzer. The gas exchange analyzer obtains the accurate metabolic rate of those subjects during that specific test while the heartrate of those subjects is collected.

A regression relating the heartrate to the energy expenditure of the user is generated. The regression is corrected to the demographic of the user (like gender, age, and weight) based on the results of the different demographics of the lab test subjects. First, we can see that there are too few variables tracked. Clearly a single variable is not enough to calculate something as complicated as the calorie expenditure of the whole body; a person's heartrate might increase watching a scary movie without burning any extra calories, while another person who is very efficient at a high metabolic activity might experience only a slight change in their heartrate despite burning a lot of energy performing that movement. Certainly, relying on only the heartrate measurement following the principles of those wearables will respectfully overestimate and underestimate the calorie expenditures.

Second, the formulated regression is not based on a scientific or physics driven models, so it is not guaranteed to provide accurate results specially when there are deviations from the nominal test conditions (change in type of sport, change in ambient conditions, change in terrain for example outdoor running compared to treadmill, or cycling uphill compared to stationary cycling, etc.) There are so many types of sports, and some movements are bound to increase your heartrate physiologically, for example, any movement that requires your hands to be higher than your head, like climbing, will spike the heartrate as the heart of the athlete tries to deliver blood to the extremities, however, that doesn't necessarily mean the body of that athlete is consuming energy equal to that consumed when the same athlete is running and having the same heartrate as that when climbing. Again, we can see that the fundamental hypothesis and approach behind the current technology is wrong. Third, the assumption that people of similar demographics operate similarly is also false: whether in age (we all have been surprised by how many 50 year old people can outperform people in their 20s, or how different some people are from their peers, or how each is efficient at an activity but not another), or in weight (a 190 lb male who is muscular is clearly consuming more than a 190 lb male who is starting to work out for the first time even if they exhibit the same heartrate doing the very same activity. Also, what about a 1701b male that does the same activity, but while wearing a 20 lb weight vest to create a bigger challenge? His heartrate shouldn't be in the regression of the 170 lb person doing the same activity without the added weight, nor should it be in the regression of either of the 190 lb fit or non-fit person), how is it fair or scientific to combine all those different cases in an empirical equation with only one variable?

Other smaller sized boutique companies have relied on measuring the carbon dioxide directly from the user's exhaled breath to approximate the calorie expenditure. This is a more accurate method compared to those mentioned above but still suffer from approximations and challenges. Such devices suffer from limiting the ability of the user to operate freely and without restrictions, also forcing breathing through the mouth while wearing a mask or a mouthpiece and thus also hindering the breathing of the user and jeopardizing his performance and thus doesn't report the real metabolic rate that he/she is capable of without having the obstruction. That is why, such devices are generally only used during the rest state to measure the rest energy consumption and approximations are then made to project those measurements for active periods later on.

### BRIEF SUMMARY

The purpose of this disclosure is to accurately measure the metabolic rate of the body, and to have it be exact for each user, i.e., avoiding regressions of a large population (like the inaccurate method currently used in the industry), and to do so in a non-intrusive method and without hindering to the user (like the accurate but bulky and inflexible gas exchange analyzer machines used in hospitals and research departments).

In summary, this is achieved by training non-intrusive and non-obstructive wearable sensors (three sensors in some embodiments) to specific body signatures of each user in an initial training phase. During this phase, the user wears the three sensors while also breathing through a gas exchange analyzer, a device that calculates the exact amount of Oxygen (O2) to Carbon Dioxide (CO2) conversion and thus the exact amount of energy expenditure. In the sensor-training phase, the user will be instructed to perform simple activities that will let his body go through a combination of different ranges of body signatures that are being tracked by the sensors.

Then, using a respiratory-system mathematical model, derived analytically and optimized according to the results of the initial training phase using assisted machine learning, the recorded body signatures can now fully represent the energy expenditure specific for that user. As a result, after the sensor-training phase, the user no longer needs the gas exchange analyzer; the user can thereafter rely on only the non-intrusive sensors/wearables to calculate his/her metabolic rate and energy expenditure during any type of activity. In some instances, the user may recalibrate their respiratory-system mathematical model by going through the sensor-training phase, as the user's lung performance can change over time.

### BRIEF DESCRIPTION OF THE SEVERAL VIEWS OF THE DRAWINGS

In the drawings, identical reference numbers identify similar features or elements. The size and relative positions of features in the drawings are not necessarily drawn to scale.

FIG. 1 is a block diagram of a system for measuring metabolic rate of a user according to an embodiment.

FIG. 2 is a diagram of the system in FIG. 1.

FIG. 3 is a mathematical representative system for measuring the metabolic rate of a user according to an embodiment.

### DETAILED DESCRIPTION

The present disclosure is directed to simultaneously solving the problem of accurate calorie expenditure calculations while maintaining the physical freedom of the user.

To do so, this system and method are configured to implement a model with variables being body signatures that can be obtained from non-obstructive sensors, such as various wearable sensors.

In bio-chemical energy breakdown, humans gain weight by eating food that contains carbon, which adds to the mass of our organic/carbon-based bodies, and humans lose weight by getting rid of the carbon, which is done by breathing it out. To produce energy that our body can use, after nutrients are simplified in the digestive system and moved into the blood, our muscle tissues break down the simple nutrients (sugar in form of glucose, fat, protein), to produce adenosine triphosphate (ATP). The breakdown, in general, requires oxygen and as a result CO2 is produced. The carbon atom in the exhaled breath came from the organic molecules that was broken down into ATP. In other words, we lose weight one carbon atom at a time. Glucose breakdown uses Oxygen and produces CO2 at a ratio of one to one (1 O2 atom/1 CO2 atom). Proteins and lipids breakdown a ratio of 1/0.8 and 1/0.7, respectively. When sufficient oxygen is not available, anaerobic glycolysis occurs, which is a very inefficient but very fast form of energy formation and thus only happens in special cases and for a very short amount of time. Anaerobic glycolysis requires zero O2. To be complete, the lactic acid formed by anaerobic glycolysis doesn't beat the system of O2 to CO2 conversion, eventually lactic acid is either modified to be used by the mitochondria with the presence of sufficient O2 resulting in producing the respective amount of CO2 atoms, or is converted back to glucose in the liver. Factoring in all the nutrients broken down, a consensus in the field is that the O2 to CO2 conversion happens with a ratio of 1 to 0.8.

Therefore, if we know now much CO2 is in the exhaled air over the original amount of CO2 from the ambient inhaled air, we can calculate how many calories are consumed instantaneously and at the time of the measurement. This is precisely how a gas exchange analyzer works; it measures the breath volume flowrate and the change in the CO2 concentration to give us the amount of energy expenditure. Some also measure O2 to give a more precise result based on the gas conversion ratio.

The system of the present disclosure is configured to identify body signatures to help determine the amount of CO2 produced by the body and to train a model of the respiratory system that incorporates those signatures to produce accurate results of calorie expenditure, such as on a user's mobile device or other display. The model will be stored in a database or memory that is either in close proximity to the user or in a remote server. As the wearable sensors on the user collect and transmit data, either wirelessly or through a direct connection, such as a user's cell phone or other handheld device, the model will collect and then output information to the user about their calorie expenditure. This can be either in real time or periodically. In some embodiments, the calculations will be performed at the end of an exercise or expenditure session. In the hands of experts and data savvy, the data can be further analyzed to provide precious information to the user, like their optimal breathing strategy (e.g. gold standards for breathing for runners is two consecutive half-breaths inhales initiated at the instant the foot hits the floor, and one full exhale. Or for swimmers, one quick full breath inhale during one stroke, followed by slow exhale over the next three strokes. However, there is no proof that this is the optimal for all users. Also no such standards are available for other sports), their body composition, their limiting factors in a certain sport (be it their metabolic conditioning or their muscular capabilities), the optimal percent of maximum effort cycling (when and how should the user pace his effort), etc. . . . the controller can incorporate data driven analysis that provides the user with the optimal strategy and training recommendations regarding all the above mentioned criteria. Moreover, the lung health and different body metrics can be used to interpret what is the sport most suitable for the user by comparing the data among all players of different sports. Also the data can be used to show the ranking of the user among the general population in each of the different metrics.

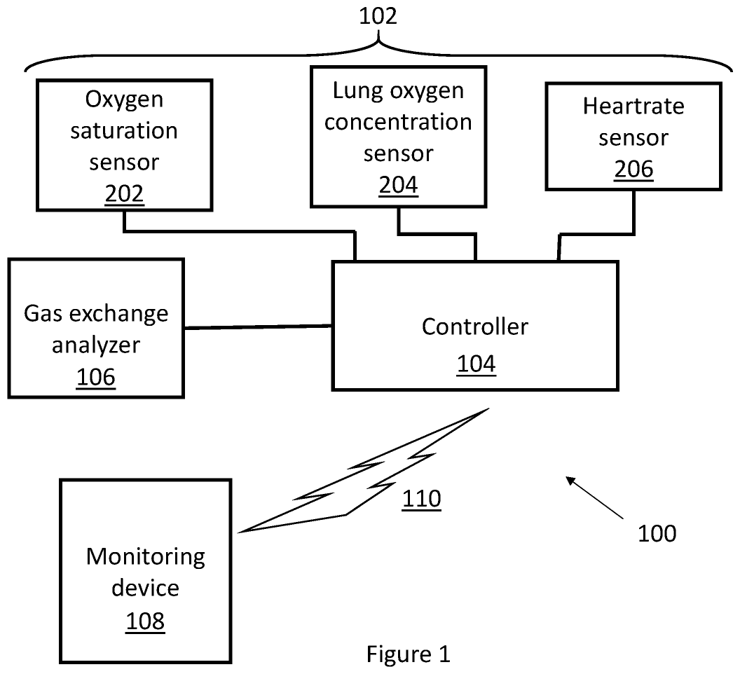

FIG. 1 is a block diagram of a system 100 for measuring metabolic rate of a user. The metabolic rate is indicative data of energy consumption of the user's body. The metabolic rate depends on an activity of the user as well as the user's physical conditions or parameters, such as the age, weight, height, gender, and their internal physical make up, such as organ properties. In particular, a property of walls of the lungs, i.e. the ability of the lungs to exchange oxygen from the lungs into the blood. The system 100 is capable of accurately measuring the metabolic rate of the user based on both activity and user's physical condition.

The system and method described herein is configured to create a user specific lung mapping for the user to track more accurate performance measurements, like energy consumption over their exercising journey. There are many ways athletes, medical professionals, and others can utilize this information to help themselves improve performance, lose weight, or otherwise improve the health of the user. Each individual user's lung can be mapped using this system and method.

The system 100 includes a plurality of sensors 102 coupled to a controller 104. The plurality of sensors 102 include wearable sensors or other type of portable sensors that are attached to or interact with the user's body. In various embodiments, the plurality of the sensors 102 includes an oxygen saturation sensor 202, a lung oxygen concentration sensor 204, and a heartrate sensor 206.

A gas exchange analyzer 106 is coupled to the controller 104. The gas exchange analyzer 106 may be coupled to the controller during a calibration process. In some embodiments, the gas exchange analyzer may be decoupled from the controller after the calibration process. The controller 104 communicates with a monitoring device 108 over a network 110. In various embodiments, the monitoring device 108 may include a smartphone, a smartwatch, a laptop, a server, or any other suitable communication, display, and processing device.

This system 100 is configured to gather real time data about the user in a first training or calibration phase and a second exercise or use phase. The first training phase includes the gas exchange analyzer 106 to set a baseline measurement of the user's amount of change in CO2 between exhaled and inhaled air, which can be calculated to provide an amount calorie expenditure.

The system and method are configured to gather information about the user's performance in the first and second phases and calculate the metabolic rate of the user using the equation below:

$$J = - \frac{L}{dx} ( O 2 blood - O 2 lung )$$

J is the amount of oxygen consumption of the body measured by the gas exchange analyzer 106 during the period of time of the calibrating the system 100, i.e. in the first training phase. The body burns around 5 kcal of energy for every Liter of O2 consumed. J is the flux, how much oxygen is being consumed. The term “O2 blood” corresponds to calculated oxygen concentration in blood that is calculated based on data of the blood oxygen saturation sensor (SPO2 is one type of such a sensor) 202 and the heartrate sensor 206. The term “O2 lung” corresponds to calculated oxygen concentration in the lung that is calculated based on data of the lung oxygen concentration sensor 204. The term “L/dx” is a constant value that depends on the lung boundary properties. For instant, “dx” represents thickness of lung membrane and “L” is a constant value that may be unique for each user dependent on the lung physical condition. Hence, the term “L/dx” is a unique value for each monitored user that is a key parameter to accurately calculate the metabolic rate. Although direct measurement of the term “L/dx” is difficult (e.g., imaging techniques such as X-ray, MRI, or ultrasonic imaging), it is calculable by the equation using the gas exchange analyzer in at least the first training phase of the system 100.

In the calibration process, J is measured by the gas exchange analyzer 106, the term “O2 blood” is calculated based on data of the oxygen saturation sensor 202 and the heartrate sensor 206, and the term “O2 lung” is calculated based on data of the lung oxygen concentration sensor 204. Hence, only the term “L/dx” in unknown during the calibration process, that can be calculated based on the equation. This term L/dx represents the properties of the lungs, where L represents a diffusion coefficient. Once the term “L/dx” is calculated, it can be assigned to the user as a signature of the user's lung boundary properties. This constant value is not changing over a long period of time (e.g., 6 months). Thus, the calibration of the system 100 may be repeated after the long period of time to update the constant value of “L/dx” of the user.

The change of the CO2 and O2 that occurs in the lungs is a result of the change of the gasses in the capillaries (blood vessels passing through the lungs and exchange air with the lung alveoli). The system is configured to measure the overall change in either the CO2 or the O2 between the outlet and the inlet of the capillaries. Blood leaving the capillaries, oxygen rich blood, has the same properties as the blood that is pumped by the heart into the aorta to go to all the body's arteries. Therefore, to know the properties of blood leaving the lungs, we can use pulse oximetry to measure the oxygen saturation (SpO2) of the oxygen rich blood with the oxygen saturation sensor 202. The oxygen saturation sensor 202 is coupled to surface of the user's skin, such as their wrist or ear, and detects a ratio of hemoglobin that is bonded to oxygen to that of unbonded hemoglobin.

The oxygen saturation sensor 202 is measured at a periphery of the user, usually a finger. The oxygen saturation sensor 202 may be a pulse oximeter, which noninvasively measures the oxygen saturation of a user's blood with a red and an infrared light source and photo detectors. The pulse oximeter includes a probe to transmit light through a translucent, pulsating arterial bed, typically a fingertip or earlobe. Oxygenated hemoglobin (O2Hb) and deoxygenated hemoglobin (HHb) absorb red and infrared light differently. The percentage of saturation of hemoglobin in arterial blood can be calculated by measuring light absorption changes caused by arterial blood flow pulsations. This is a transmissive method. An alternative method is a reflective method where a transmitter and receiver are on a same side of the user's skin, such as in a watch on a user's wrist.

With the blood entering the capillaries, which has the same properties as the blood returning to the right atrium of the heart, we can determine the mixed venous oxygen saturation (SvO2) to provide the percent oxygenation of the blood returning to the right side of the heart. This reflects the amount of oxygen “left over” after the tissues consumed what they need.

A simple mathematical equation can give us the result of oxygen consumed by the body: O2 consumed=volume flowrate of the blood x hemoglobin concentration per blood volume×(SpO2−SvO2).

Over different time periods the concentration of hemoglobin in the blood of a specific user does not change. Therefore, in the above equation, hemoglobin concentration is a constant property specific for each user. Blood volume flowrate is a function of the heartrate, heart stroke, and the capillary cross section area (some aggregate mean diameter/cross section of all the capillaries). The latter two properties are very hard to measure, however, we know that they are also fixed properties of the specific user. Therefore, the equation above could be written with the measurable body signatures (heartrate, SpO2, SvO2) as variables, and the fixed body properties as fixed parameters/constants: O2 consumed=constants×f(heartrate)×(SpO2−SvO2). Where f is a function to be determined during the sensor training phase along with the overall value of all the constants.

In various embodiments, the gas exchange analyzer 106 may be temporarily coupled to the controller 104 for calibrating the system 100. During a calibration mode or first training phase, the controller receives indicative data from the plurality of sensors 102 in addition to data from the gas exchange analyzer 106. The data from the gas exchange analyzer 106 may indicate oxygen consumption of the user during a specific period of time. In this condition, the controller 104 may calculate the metabolic rate of the user based on the oxygen consumption of the user's body during the period of time which the gas exchange analyzer 106 is worn by the user. The gas exchange analyzer accurately measures conversion of the inhaled oxygen (02) into the exhaled carbon dioxides (CO2). This measured conversion accurately estimates a value of organic material that has been burned by the body of the user that is corresponding to the metabolic rate of the user. Although the gas exchange analyzer 106 calculates the metabolic rate of the user during the period of time, it is not practical to be worn by the user for a long time. The gas exchange analyzer 106 may only be worn by the user during the specific period of time for calibrating the system 100, i.e. the first phase.

The lung oxygen concentration sensor 204 is configured to determine a volume of air inhaled by a user. It is noted that each of the measurements will be collected with a time stamp so that the different measurements from the different sensors can be collated together to reflect the user's performance during a specific moment in time or over a time period.

The lung oxygen concentration sensor 204 may measure the breathing volume of the user by indication of the rib cage deformation of the user's body. For instance, the lung oxygen concentration sensor 204 may include a wearable device that wraps around a rib cage of the user and includes a resistive or pressure sensor. The device may be an adjustable belt to be worn by the user and tightened around chest of the user so that the expansion of the chest during breathing can be monitored as a force applied to the pressure sensor on the belt. The pressure sensor may be a capacitive sensor that produce a voltage change based on the expansion of the chest during the breathing. Thus, indicative data can be a voltage variation to be transmitted to the controller 104 by the lung oxygen concentration sensor 204. The controller 104 may be configured to perform signal processing on the received data from the lung oxygen concentration sensor 204, to calculate the breathing volume of the user based on the voltage variation.

Alternatively or in addition, an acceleration sensor may be coupled to the belt. The acceleration sensor measures movement of the chest of the user. In response, an indicative signal can be analyzed by the controller 104 to calculate the breathing volume of the user based on the movement of the chest. For instance, the acceleration sensor may be MEMS (micro-electromechanical system) high-resolution capacitive accelerometers. The sensor will be calibrated to be able to identify which movements correspond to which volume of air for the user. This can be performed in the first phase, the training phase.

The belt may be resistive stretch sensors including conductive material and polymer. In this condition, one or more belts can convert deformation of the rib cage during breathing of the user into a change of resistances of the one or more belts. In some examples, the one or more belts may be integrated into the clothing. The belt may also include piezoelectric devices, in which the deformation of the belt creates electrical signal as the indicative data for measuring breathing volume of the user.

In another embodiment, the breathing volume of the user may be measured directly from the inhaled and exhaled air pressure during breathing. For instant, the lung oxygen concentration sensor 204 may include an electronic device coupled to a mask to form a wearable sensor. In this condition, the electronic device may include pressure transducers and a fan that operates in a forced oscillation technique (FOT) mode. In this embodiment, the indicative signal may be a differential signal based on the inhaled and exhaled air pressure during breathing. The indicative signal may be transmitted to the controller 104 and analyzed to calculate breathing volume of the user.

In some embodiments, a wearable sensor may include an acoustic sensor to measure breathing volume of the user based on the lung or throat or esophagus sounds. In this embodiment, the acoustic sensor may be a microphone positioned close to the nose, mouth, throat, and suprasternal notch of the user. In some embodiments, the indicative data transmitted to the controller 104 includes sounds that can be classified to eating, drinking, speaking, laughing, coughing, and breathing sounds. In this condition, the controller 104 may filter unwanted sounds and calculate the breathing volume of the user base on the breathing sounds.

The accurate oxygen concentration in the lung of the user may relate to the environmental conditions in addition to the breathing volume of the user. For enhancing accuracy of the calculation, in some embodiments, the lung oxygen concentration sensor 204 may also include an environmental sensor. In this condition, the controller 104 combines the indicative data of the breathing volume of the user with the environmental data of the environmental sensor. In various embodiments, the environmental sensor may include a barometer which measures atmospheric pressure, and consequently elevations of the user. In some alternative embodiments, the environmental sensor may include GPS to calculate elevation of the user or use the user's phone location services. The environmental conditions, such as elevation and atmospheric pressure may directly affect the relation between the rib cage deformation and breathing volume of the user. Hence, a combination of the environmental data with the indicative data of the breathing volume of the user, provides more accurate calculation of the oxygen concentration in the lung of the user. In various embodiments, the environmental sensor may also include one or more of temperature sensors, humidity sensor, gas sensors, and combination thereof. In summary, the term O2 Lungs is volume of inhaled air plus the atmospheric condition.

In some embodiments the blood oxygen saturation sensor 202 operates simultaneously with the heartrate sensor 206, such as being housed in a same wearable device, like a smart watch. In this condition, the controller 104 receives indicative data from the oxygen saturation sensor 202 and the heartrate sensor 206 corresponding to a real time condition of the user. The controller 104 is configured to calculate an oxygen concentration of blood for the user, based on the data received simultaneously from the oxygen saturation sensor 202 and the heartrate sensor 206. A pulse oximeter may operate as a combination of the oxygen saturation sensor 202 and the heartrate sensor 206.

The controller 104 may be configured to calculate the oxygen concentration in blood of the user under monitoring, based on received data from the heartrate sensor 206 which indicates flowrate of blood in body of the user, and data from the oxygen saturation sensor 202 which indicates oxygen saturation level in hemoglobin of blood. In this condition, a function may be predetermined in the controller 104, wherein the function has two inputs from the oxygen saturation sensor 202 and the heartrate sensor 206, and one output as the oxygen concentration in blood of the user under monitoring.

By calculating oxygen consumption of the body of the user, the system 100 is capable of accurately calculate metabolic rate of the user. For calculating oxygen consumption of the body, breathing volume of the user can be measured that indicates oxygen concentration in lung of the user. The lung oxygen concentration sensor 204 may operate simultaneously with the oxygen saturation sensor 202 and the heartrate sensor 206. In this condition, the lung oxygen concentration sensor 204 measures and transmits indicative data of the oxygen concentration in lung of the user to the controller 104. The controller 104 may calculate the oxygen consumption of the body based on the received data simultaneously from the lung oxygen concentration sensor 204, the oxygen saturation sensor 202, and the heartrate sensor 206.

After calibrating the system 100, the gas exchange analyzer 106 can be decoupled from the controller 104 and the term “J” is calculated based on the real time measurement of the term “O2 blood” and the term “O2 lung” of the equation with the controller, the monitoring device, or a remote server (not illustrated). During the calibration process, different conditions may include different activities of the user, such as different exercises, during resting time, and during sleeping time. In some embodiments, the equation may be calculated for different environmental conditions by generating various data corresponding to the different environmental conditions, such as different atmospheric pressures, different humidity conditions, and different environmental temperatures. In this condition, the different data may be stored in a memory of the controller 104 to be analyzed during the calibration process. In some embodiments, a curve fitting process may be performed to calibrate the values of the equation corresponding to the different conditions of the recording data.

In some embodiments, the different recorded data may be used to train a Machine Learning system, where the Machin Learning system may accurately estimate the metabolic rate of the user based on the measurements of the term “O2 blood” and the term “O2 lung” of the equation in different conditions. The Machin Learning system may generate a first indicative data of the metabolic rate based on the measured “O2 blood” and “O2 lung” when the user is doing an exercise such as biking, while generating a second indicative data of the metabolic rate based on the same measured “O2 blood” and “O2 lung” when the user is doing another exercise such as hiking, while the second indicative data is different than the first indicative data. Thus, the system 100 is capable of dynamically measuring real time metabolic rate of the user by considering different parameters that make the measurement more accurate compared with conventional methods. The different parameters may include, environmental conditions, physical conditions of the user, and type of the activity of the user during the operation of the system 100.

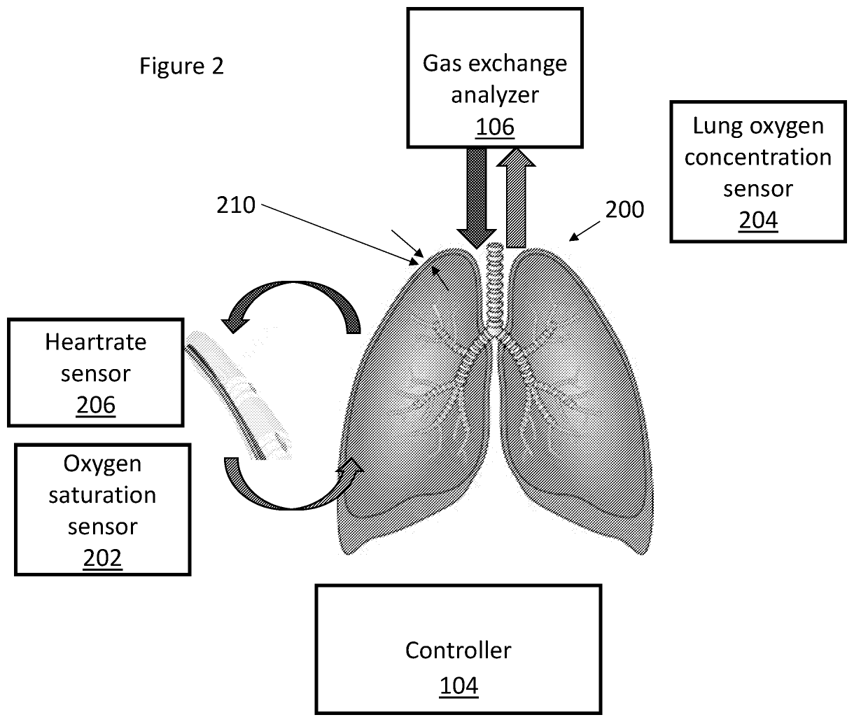

FIG. 2 is a diagram of the system in FIG. 1 with lungs illustrated to emphasize different interactions. The gas exchange analyzer 106 is coupled to the input and output of the lung 200 of the user (e.g., reparatory passages), which is breathing way of the user through mouth and nose. The gas exchange analyzer 106 may include a mask to be worn by the user to measure inhaled oxygen (O2) and the exhaled carbon dioxides (CO2) of the lung 200. The monitoring data from the gas exchange analyzer 106 can be used for training a Machine Learning system or performing a curve fitting to retrieve boundary properties of lung 200. As described in equation, the term “L/dx” is a constant value that depends on the lung boundary properties. L/dx does not change day over day, but will improve over time, can track lung performance over time. The term “dx” represents thickness of lung membrane that is shown with a dimension 210 of lung 200 in FIG. 2. In this condition, “L” is a constant value that is unique for each user and depends on the physical condition of lung 200. The physical condition may be related to size, volume, and stiffness of lung 200 in addition to health condition, age, and gender of the user. Hence, the monitoring data from the gas exchange analyzer 106 provides information about real physical condition of lung 200 that can be used for real-time estimation of the metabolic rate measurement by the system 100 described in FIG. 1. The real-time measurement of the metabolic rate can be performed without needing to use the gas exchange analyzer 106.

In some embodiments, the oxygen concentration of lung 200 is measured by the lung oxygen concentration sensor 204. The lung oxygen concentration sensor 204 may be one of the various sensors described in FIG. 1, such as a pressure sensor coupled to a belt. In some embodiments, the measured data from the lung oxygen concentration sensor 204 is transferred into the controller 104 through a wired communication link. Alternatively, the measured data may be transferred to the controller 104 by a wireless communication link such as Bluetooth or near-field communication (NFC). The controller 102 may be integrated in a wearable device such as a smartwatch. In some alternative embodiments, the controller may be in the monitoring device 108 described in FIG. 1. In this condition, the monitoring device 108 may be a smartphone which wirelessly communicates with the lung oxygen concentration sensor 204.

In various embodiments, the heartrate sensor 206 and the oxygen saturation sensor 202 may be integrated in a same wearable device that includes the controller 104, e.g., a smartwatch. In an alternative condition, the heartrate sensor 206 may be a separate wearable sensor than the oxygen saturation sensor 202. In some embodiment, the heartrate sensor 206 and the oxygen saturation sensor 202 may wirelessly communicate with the controller 104, while the controller 104 is in the monitoring device 108, e.g., a smartphone. The controller 104 may include a non-transitory readable memory to record data during the calibration process. The controller 104 calibrates the system 100 based on the recorded data to operate without need to the gas exchange analyzer 106 after the calibration. In some embodiments, the calibration process includes producing an algorithm to accurately predict metabolic rate of the user based on measurement data from the lung oxygen concentration sensor 204, the heartrate sensor 206, and the oxygen saturation sensor 202 in real-time.

In another embodiment, the recorded data may be used to train a Machine Learning system. In this condition, the Machine Learning system is capable of predict metabolic rate of the user for the conditions that even was not expected during the calibration process. For example, the recording data may include only some limited number of exercises, such as hiking, biking, and running. However, the Machine Learning system is capable of predicting metabolic rate of the user for other exercises such as swimming, that was not included in the calibration process. For increasing accuracy of the Machin Learning system, one or more input data may be used in addition to the outputs of the sensors. For example, the one or more input data may include activity environment, muscles engaged in the activity, aerobic activity, and a combination thereof.

In some embodiments, the plurality of sensors 102 may be integrated in a wearable device such as a smartwatch. In addition, each sensor of the plurality of sensors 102 may be separately attachable to the human body. In some embodiments, the plurality of sensors 102 may be integrated by the controller 104 inside a portable device, e.g., a smart watch. In this condition, the metabolic rate of the user is measured by the portable device and transmitted to the monitoring device 108 over the network 110. The monitoring device 108 may include a display to notify the user about the metabolic rate received from the portable device. In various embodiments, the metabolic rate may be displayed in energy burning unit such as calorie over time.

The network 110 may be wired or wireless. The wireless network may include short-range communication such as Bluetooth and near field communication (NFC) networks, or long-range communication such as Wi-Fi and cellular networks.

In the second, use phase, the oxygen saturation sensor 202, the lung oxygen concentration sensor 204, and the heartrate sensor 206 are coupled to the user's body and coupled to the controller to periodically or in real-time gather information about the user.

As noted above, the oxygen saturation sensor 202 may include an arterial oxygen saturation (SpO2) sensor. In general, SpO2 or peripheral capillary oxygen saturation, represents oxygen saturation which shows how many RBC hemoglobin have bonded to an oxygen molecule. Not all RBC hemoglobin will bind an oxygen molecule during respiration, especially during a shortage in oxygen supply. The percentage of red blood cells that have made this chemical bond during breathing is the measured level of oxygen saturation. The normal SpO2 range is 90-100% and a lower percentage will indicate there is a critical imbalance in the oxygen supply and demand. Currently, pulse oximetry is the standard to measure SpO2.

As noted above, SpO2 monitoring with pulse oximetry typically includes two lights pass through the pulse oximeter clamp on a finger, toe, or ear, e.g., infrared and red lights. Increasing of oxygen-bound hemoglobin increases infrared light absorption. Inversely, if there is no oxygen-bound hemoglobin, only the red light is absorbed. Thus, a percentage of oxygen saturation is measured by calculating an absorption ration between the infrared light opposed to the red light. The result giving the measurement of oxygenation as a percentage.

The output of the training phase may be a number or range of numbers and datasets that represent the user's flux, J, or gas exchange efficiency of the lungs (L/dx), which is about the collective performance of the lungs of the user. The flux is stored in memory in at least the controller, the monitoring device, or the remote server as the specific lung mapping data of that user. During the second phase, this flux is used in conduction with the other sensors worn by the user during exercise or other activities to provide information about a more accurate representation of energy consumption. The memory and processing unit in one of the controller, the monitoring device, or the remote server stores the equation and software configured to determine the energy expenditure based on the stored flux value of the specific user and the other data being collected by the worn sensors, such as the heartrate sensor, the lung oxygen concentration sensor, and the oxygen saturation sensor.

The system can be provided to the user as a kit that includes a heartrate sensor, an oxygen sensor, and the lung volume or concentration sensor. The heartrate sensor and oxygen sensor may be in a single device to be coupled to the user, such as in a watch. The watch may then be coupled to the lung volume detector, like a band around the ribs, wirelessly or through a wired connection.

Alternatively, the heartrate sensor and oxygen sensor in the watch may also include a sensitive microphone configured to pick up and detect an inhale and exhale rate of the user to determine lung oxygen concentration. Alternatively, the heartrate sensor and the oxygen sensor may be integrated within the belt or rib cage band device, instead of being distinct devices.

The system is configured to determine metabolic rate for the user at a point in time, such as periodically during exercise, either a selected interval or by selection by the user through an interface such as display screen of the watch or monitoring device. Alternatively, this can be generated at the end of an exercise session by either selection by the user or automatically after the system has detected no further exercise movements for a time period.

The user can understand more information about their body composition and energy expenditure with this system and method. This system can gather information about baseline energy expenditure, such as by monitoring during the user's sleep after the first phase where the only energy expenditure is tied to base line organ needs.

This system will allow a user to determine how much they burned during exercise to then determine how much to consume to recover. The user will have information about how much they have stressed their body and to understand how much recovery may be needed.

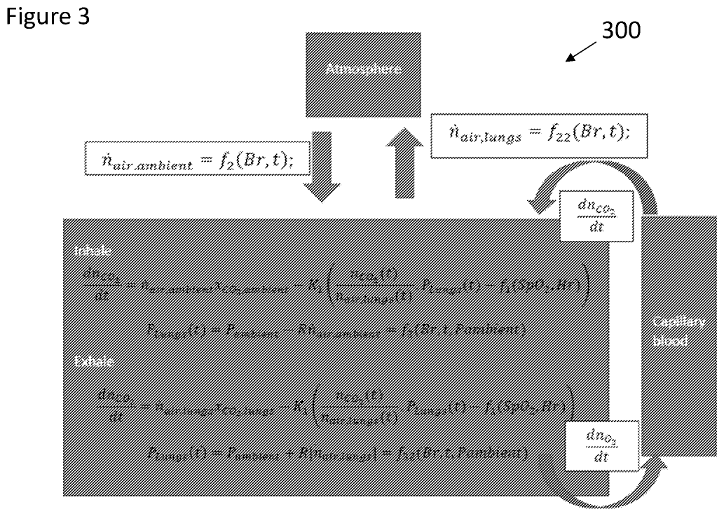

Above we presented the simple general idea of the mathematical lung model. However, the detailed model is a time dependent, i.e. the above equation holes for each instant in time, and to find the overall aggregate energy expenditure, we must solve the differential equation that is described in more details below. FIG. 3 is a mathematical representative system 300 for measuring the metabolic rate of a user. The mathematical representative system 300 calculates metabolic rate of the user based on a conversion of oxygen into carbon-dioxide (CO2) with lung of the user. An exchange of CO2 gas can be calculated based on a derivative function of a gas pressure inside the lung and gas pressure of ambient of the user. More accurate measurement is possible by considering information about oxygen saturation of blood and heartrate of the user. The equation below represents relation between the CO2 exchange and gas pressure inside lung of the user during inhalation.

dnCO₂dt=\dot{n}air,ambientxCO₂,ambient−K1(nCO₂(t)nair,lungs(t)·PLungs(t)−ƒ1(SpO2,Hr))

\dot{n}air,ambient=dVlungsdtρair,ambeintMair,ambient=ƒ2(Br,t);

PLungs(t)=Pambient−R\dot{n}air,ambient=β(Br,t,Pambient)

The term n is the number of mole, K1 is a constant, rho is density, M is molar mass, t is time, P is gas pressure, and R is the resistance to the air flow created by the reparatory passages.

Note the use of f3 (once trained) can also incorporate the effect of water vapor evaporated from the lungs and added the air inside the lungs, resulting in decreasing the partial pressure of CO2 (and O2) in the lungs. n_{air,lungs}(t)=Bv(t)ρ_{air,lungs}(t)M_{air,lung}(t)+∫(dnco₂dt−dno₂dt)dt

Where rate of change of O2 is 0.8 that of CO2

With initial conditions:

nair(0)=moles of air in deadspace, nco2(0)=end tidal moles of CO2=g(hr,spo2,br).

During exhalation there are small changes to the above equations since the direction of flow changes and the source of the air exiting is the air inside the lungs:

dnco₂dt=\dot{n}_{air,lungs}xco_{2,lungs}−K₁(nco₂(t)n_{air,lungs}(t)·P_{Lungs}(t)−ƒ₁(SpO₂,Hr))

\dot{n}_{air,lungs}=dV_{lungs}dtρ_{air,lungs}(t)M_{air,lungs}(t)=ƒ₂₂(Br,t);

P_{Lungs}(t)=P_{ambient}+R|\dot{n}_{air,lungs}|=ƒ₃₂(Br,t,Pambient)

Calorie expenditure=4.8008/0.8 (kcal/letter of CO2)*VCO2 produced (letters)

In fact, the coefficient above ranges between 4.6862/0.8 to 5.0468/0.8 kcal/letters of CO2 depending on the source of carbon atoms (glucose or protein or fat). The exact value of the coefficient can be picked according the diet of each user, or from the gas exchange analyzer of 30 phase1 if a simultaneous CO2 and O2 gas analyzer is used.

It is noteworthy to say that we can simply write calorie expenditure=ftotal(hr, spo2, br, by) and let the optimizer and the neural network figure out the best fit, since we just proved that these are the measurable variables that fully define this process. However, writing the scientific equations 1-helps the neural network in coming up with the optimal fit, 2-eliminates the many other possible functions that could fit these criteria within the same data set witnessed in the training process, but would not be proper to extrapolate outside the range of that specific data.

The embodiments of the present disclosure include employing the training process to teach the sensors how to relate their readings to the exact calorie expenditure. The user would first wear three sensors, heartrate sensor, SpO2 sensor, and SvO2 sensor, while breathing through a gas exchange analyzer. Going through a range of variations in the body signatures we can find out the single function above, f, and relate the oxygen consumed by the body to the measured signatures. After the training process is done, the user can wear the sensors without the need for the gas exchange analyzer and be free to perform any activity while accurately calculating his energy expenditure based on the scientific model just created and the functions just calibrated.

We mentioned above that this is the simplest implementation of the idea and that is because of the following: While heartrate sensors and SpO2 sensors are very easily available and very widespread in the health and health and fitness industries, a non-intrusive non obstructive SvO2 measurement device is not yet available. Non-intrusive SvO2 rely on approximation from some easier accessible veins that are prompted to pulsate (you can read about the importance of pulsation in oximetry, as a method to eliminate the constant offset in readings that the different body tissues create, to obtain the pure reading from the blood oxygen concentrations only). Or approximated form the jugular vein that is very exposed on the neck; while a jugular measurement may give an idea of a person's health it is hard to generalize its uses to metabolic rate especially under physical activities where muscle tissues consumption is hard to correlate with head energy consumption. So, if we follow any good approximation or exact calculation of the SvO2, which is not yet the case, our quest would be over and we would be able to use three non-intrusive sensors to measure our energy expenditures. But since SvO2 measurements aren't as easy or as accurate as we wish with the current technology, we must move to a more elaborate and practical scientific model of the respiratory and cardiac systems.

There are several different breathing models in literature with varying complexity that can be used, but require changing the control variables of those models to the body signature variables that wearable sensors can measure. However, next we will derive one accurate model of the respiratory system to show how the selected variables fully define the problem and are sufficient to accurately predict the metabolic rate.

Consider the breathing model where we track the mass of O2 into and out of the lungs:

1—A certain amount of air from the ambient is inhaled into the lungs bulk mass movement into the lungs a. The amount of O2 in the lungs increase according to VO2=VAir, ×Xo2, ambient (V is the volume of inhaled air and XO2 is the molar fraction of O2 in that air)

2—Air in the lungs exchange gasses with the blood capillaries across the alveoli and capillary wall boundary gas diffusion across a boundary where O2 enters the blood and CO2 enters the lungs

a. O2 amount in the lungs changes according to the mass diffusion equations JOP2=−DA dPO2 (lungs-blood)/dx where x is the alveoli-capillary membrane, and D is the diffusion coefficient representing the properties of the membrane, and A is the area of that membrane. Notice that these three parameters are physiological constant specific for each user. This is very important for the theory of our respiratory model calibration later. dPO2(lung-blood) is the difference between the concentration of O2 in the lungs to that in the blood which is the driving force of the diffusion and the only variable in that equation.

3—A certain amount of Air is exhaled from the lungs into the atmosphere bulk movement out of the lungs VO2=−VAir, exhaled x XO2, exhaled

Now let us see how each of these steps can be tracked

1—Inhalation: the movement of the thoracic cage can be related to the amount of air inhaled. We utilize a displacement sensor, for example an elastic strap around the chest that measures the change in the circumference of the chest vs time. Such displacement sensors can be based on resistive or capacitive or piezoelectric sensors etc. or we can use a sensor that measures the muscles electric signal, in this case it would be the electric signal from the thoracic case muscles and/or the lung diaphragm, or we can use an acoustic respiration rate sensor. We denote the outputs of this sensor as breathing rate and breathing volume (Br, By)

2—Air exchange equation can be elaborated into a finite version as opposed to the differential infinitesimal version. JO2=rate of change of O2 in the lungs=−DAdPO2 (lungs−blood)/dx=−DA (PO2lungs-PO2blood)/deltaX a. The concentration of the O2 in the blood varies as blood moves in the capillaries and exchanges gasses with the lungs, however, a mean amount of O2 can be used in the equation (PO2bloodmean). The mean concentration of O2 in the capillaries depends on the initial concentration of O2 at the inlet of the capillaries, and on the amount of blood flowing through the capillaries. Increasing the O2 concentration in the blood entering the capillaries, all else equal, increases the amount of mean O2 in the capillaries and vice versa. Also, all else equal, increasing the amount of blood flowrate in the capillaries results in a smaller change in the concentration of O2 in the blood and thus a smaller mean value of Po2blood. But as explained in the simple model above, the amount of blood flowrate is a direct relation to the heartrate of the user. Therefore, increasing the heart rate of the user, decreases the mean O2 concentration in the blood and vice versa. The heartrate is easily measured, but what about the O2 concentration entering the capillaries. Theoretically, we can use optical sensors across the skin to measure the concentration of the O2 in veins (SvO2), however as explained above, measuring SvO2 has many challenges.

b. Moving on, PO2bloodmean=(PO2blood,exit×Vblood−JO2)/Vblood (essentially to find the mean partial pressure of O2 we find the amount of O2 present in the exit of the capillaries and we subtract the amount of O2 that was added by the lungs to the blood (JO2), then we normalize by the blood volume). Simply put, we now can define the partial pressure of oxygen in the blood in terms of SpO2 and the heartrate as such: PO2bloodmean=f(SpO2, Hr), f here is a new function, and again we do not need to know now the exact form of the functioi-L since we will be using sensor training to find the mathematical expression that defines those relations specifically for each user.

c. Now looking at the control volume of the lungs, we can write the law of conservation of mass, or conservation of O2 molecules to produce the differential equation describing the change of oxygen as a function of time within each inhale and exhale.

d. Combining the equations from this section we get: rate of change of O2 in the lungs=Constants (PO2lungs−f(SpO2,Hr)). The constants and the new function f will be determined during the training phase of the sensors related to the specific user.

3—Exhalations: similar to inhalation, can be tracked by the thoracic cage movement.

Now we can construct the differential equation defining the O2 consumption (or CO2 production). We will switch to tracking CO2 instead of O2 because it will be easier when we want to implement the initial conditions to the differential equation that will be derived next. FIG. 3 represents the control volume of the lungs:

dnco₂dt=\dot{n}_{air,ambient}xco_{2,ambient}−K₁(nco₂(t)n_{air,lungs}(t)·P_{Lungs}(t)−ƒ₁(SpO₂,Hr))

\dot{n}_{air,ambient}=dV_{lungs}dtρ_{air,ambient}M_{air,ambient}=ƒ₂(Br,t);

P_{Lungs}(t)=P_{ambient}+R\dot{n}_{air,ambient}=ƒ₃₂(Br,t,Pambient)

Where n is the number of mole, K1 is a constant, rho is density, M is molar mass, t is time, P is gas pressure, and R is the resistance to the air flow created by the reparatory passages.

Note the use of f3 (once trained) can also incorporate the effect of water vapor evaporated from the lungs and added the air inside the lungs, resulting in decreasing the partial pressure of CO2 (and O2) in the lungs. n_{air,lungs}(t)=Bv(t)ρ_{air,lungs}(t)M_{air,lungs}(t)+∫(dnco₂dt−dno₂dt)dt

Where rate of change of O2 is 0.8 that of CO2

With initial conditions:

nair(0)=moles of air in deadspace, nco2(0)=end tidal moles of CO2=g(hr,spo2,br).

During exhalation there are small changes to the above equations since the direction of flow changes and the source of the air exiting is the air inside the lungs:

dnco₂dt=\dot{n}_{air,lungs}xco_{2,lungs}−K₁(nco₂(t)n_{air,lungs}(t)·P_{Lungs}(t)−ƒ₁(SpO₂,Hr))

\dot{n}_{air,lungs}=dV_{lungs}dtρ_{air,lungs}(t)M_{air,lungs}(t)=ƒ₂₂(Br,t);

P_{Lungs}(t)=P_{ambient}+R|\dot{n}_{air,lungs}|=ƒ₃₂(Br,t,Pambient)

Calorie expenditure=4.8008/0.8 (kcal/letter of CO2)*VCO2 produced (letters)

In fact, the coefficient above ranges between 4.6862/0.8 to 5.0468/0.8 kcal/letters of CO2 depending on the source of carbon atoms (glucose or protein or fat). The exact value of the coefficient can be picked according the diet of each user.

It is noteworthy to say that we can simply write calorie expenditure=ftotal(hr, spo2, br, by) and let the optimizer and the neural network figure out the best fit, since we just proved that these are the measurable variables that fully define this process. However, writing the scientific equations 1-helps the neural network in coming up with the optimal fit, 2-eliminates the many other possible functions that could fit these criteria within the same data set witnessed in the training process, but would not be proper to extrapolate outside the range of that specific data.

The user will wear the three sensors, while breathing through a gas exchange analyzer. The gas exchange analyzer calculates the speed of air passing through a fixed duct, to obtain the instantaneous flowrate, air flowrate as a function of time. Meanwhile, it will also measure the concentration of CO2 in the exhaled stream (using spectrography) to obtain the co2 concentration as a function of time. From there the total amount of CO2 can be obtained (integral of co2 concentration with respect to volume of air). We utilize optimization and neural network to find out the relations between the measured variables and the output of the gas analyzer based on the utilized scientific model of the lungs like the one constructed above. Essentially the AI helps figure out all the user specific body constants and compositions. This work will be further elaborated into establishing new heath criteria and characteristic information for the user based on the obtained values of the constants of the scientific model of the lungs.

Finally, after the sensors are trained to that specific user, the user can rely on only those non-intrusive and non obstructive sensors to estimate his calorie expenditure throughout any physical activity

In the above we present a method for training wearables to accurately measure the energy expenditure, however, the idea can be used to obtain accurate estimation of any body characteristic using an initial sensor training phase with a dedicated measuring device that may be restrictive, so that later, after the training phase is over, let go of the dedicated measuring device and allow the user to be unrestricted while still measuring the body characteristic using just the wearables. For example, the procedure presented here could be used to train the pulse oximetry of a vein. Pulse oximetry requires pulse in order to remove the offset caused by the tissues and just measure the oxygen concentration in the blood stream, but unlike arteries veins do not pulsate. Therefore, initially the vein would be cuffed at two different locations to induce the pulse in the vein in between the two cuffing locations. A pulse oximetry will measure the body signatures over a range of blood flowrate and pressure and oxygen concentration. After the oximetry sensor is trained on the contribution of the blood flow and of the tissues, the user can let go of the cuffing process and rely on only the trained oximeter to obtain the required value of vein oxygen concentration.

The present disclosure is directed to a system that includes one or more primary sensors configured to measure concentration of oxygen in blood of human body; one or more secondary sensors configured to measure concentration of oxygen in lung of the human body; a gas exchange analyzer configured to measure oxygen consumption by the human body; and a controller configured to: calculate lung boundary properties based on the measured data from the primary and secondary sensors and the gas exchange analyzer; generate an algorithm corresponding to the lung boundary properties, the algorithm generates oxygen consumption data based on the lung boundary properties and measured data from the primary and secondary sensors; generate indicative data of metabolic rate based on the algorithm; and transmit indicative data of metabolic rate to a monitoring device.

The system includes the primary sensors being a heartrate sensor and an arterial oxygen saturation (SpO2) sensor. The system includes the secondary sensors being a breathing volume sensor. The breathing volume sensor includes a wearable sensor coupled to a belt, the belt is positioned around rib cage of the human body. The wearable sensor includes a pressure sensor. The controller is further configured to train a Machine Learning system based on the algorithm, the Machine Learning system calculated the metabolic rate based on the measured data from the primary and secondary sensors and without the data from the gas exchange analyzer.

The monitoring device includes a smartphone wirelessly coupled to the controller. The primary and secondary sensors are integrated in a wearable device, the wearable device is calibrated based on the algorithm corresponding to a wearer at the time of calibration. The controller is further configured to train a Machine Learning system during the calibration, and generate the metabolic rate based on the trained Machine Learning system during an operation mode, the gas exchange analyzer is in use during the calibration and is disabled during the operation mode.

An alternative representation of the present disclosure includes oxygen flux from the air in the lungs to the blood that can be described by applying Flick's laws of diffusion, as shown by equation (1) below:

$${J}_{{O}_{2}} ( t ) = - D \frac{[ {O}_{2} blood ] ( t ) - [ {O}_{2} lungs ] ( t )}{dx} , \qquad ( 1 )$$

where J is the Flux, mole per unit area per time; D is the diffusion coefficient; dx is the thickness of the membrane/lung-capillary wall; [O₂blood] and [O₂lungs] are the concentration/partial pressure of oxygen in the blood and the lungs respectively.

Therefore, the total amount of O2 transferred in mole at an instant of time t is shown by equation (2) below:

$${n}_{{O}_{2} diffused} ( t ) = {AJ}_{{O}_{2}} ( t ) = - DA \frac{[ {O}_{2} blood ] ( t ) - [ {O}_{2} lungs ] ( t )}{dx} , \qquad ( 2 )$$

where A is the total area of the lungs. The parameters D, A, and dx, are user specific constants, their values depending on the size and adaptation of the lungs, and thus their values are fixed across an extended period of time. When a significant time passes and the user have had an improved or regressed adaptation, then the values of those constants can increase or decrease.

Oxygen mole balance in the lungs is shown by equation (3) below:

$$\begin{matrix}\frac{{dn}_{{O}_{2} lungs}}{dt} = {O}_{{2}_{inhale}} ( t ) - {n}_{{O}_{2} diffused} ( t ) \\ = {O}_{{2}_{inhale}} ( t ) + dA \frac{[ {O}_{2} blood ] ( t ) - [ {O}_{2} lungs ] ( t )}{dx} \\ = {O}_{{2}_{inhale}} ( t ) + dA \frac{[ {O}_{2} blood ] ( t )}{dx} - dA \frac{[ {O}_{2} lungs ] ( t )}{dx}\end{matrix} , \qquad ( 3 )$$

where O₂inhale is the amount of inhaled oxygen, it is positive during inhalation, and negative during exhalation.

The inhaled oxygen, O₂inhale (t), is a function of the breathing rate, BR(t), obtained from the breathing volume sensor, at the concentration of oxygen in the ambient (21% at sea level, and lower with increasing elevation, obtained from an altitude sensor, GPS or phone location service) according to equation (4) as shown:

O₂_{ᵢₙₕₐₗₑ}(t)=[O_{2ambient}]BR

The concentration/partial pressure of oxygen in blood, [O2blood](t), is a function of the oxygen arterial saturation SpₐO₂, obtained by the pulse oximetry sensor, and the blood flowrate, Hr(t), obtained from the heartrate monitor or sensor. Such a relation is found in FIG. 1 of Collins J A, Rudenski A, Gibson J, Howard L, O'Driscoll R. Relating oxygen partial pressure, saturation and content: the haemoglobin-oxygen dissociation curve. Breathe (Sheff). 2015 September; 11(3):194-201. doi: 10.1183/20734735.001415.

SpₐO₂ is measured at the oxygenated blood after it has left the lungs. Therefore, the average/mean value of the oxygen partial pressure in the blood at the location of the lungs is expressed as

$$[ {O}_{2} blood ] ( t ) = \frac{[ {O}_{{2}_{{Sp}_{a}}} ] Vblood ( t ) - {n}_{{O}_{2}} ( t ) / 2}{Vblood ( t )} ,$$

where [O_{2Spa} is the concentration of oxygen in the arteries obtained from the SpₐO₂. For brevity we are not going to elaborate here. Vblood is the blood flowrate which is obtained from the heartrate sensor.

$$\begin{matrix}[ {O}_{2} blood ] ( t ) = \frac{[ {O}_{{2}_{{Sp}_{a}}} ] Vblood ( t ) + DA \frac{[ {O}_{2} blood ] ( t ) - [ {O}_{2} lungs ] ( t )}{2 dx}}{Vblood ( t )} \\ = \frac{[ {O}_{{2}_{{Sp}_{a}}} ] Vblood ( t ) + DA \frac{[ {O}_{2} blood ] ( t )}{2 dx} - DA \frac{[ {O}_{2} lungs ] ( t )}{2 dx}}{Vblood ( t )}\end{matrix} \\ [ {O}_{2} blood ] ( t ) [ Vblood - DA \frac{1}{2 dx} ] = [ {O}_{{2}_{{Sp}_{a}}} ] Vblood ( t ) - DA \frac{[ {O}_{2} lungs ] ( t )}{2 dx} \qquad ( 5 )$$

$$[ {O}_{2} blood ] ( t ) = \frac{[ {O}_{{2}_{{Sp}_{a}}} ] Vblood ( t ) - DA \frac{[ {O}_{2} lungs ] ( t )}{2 dx}}{Vblood - DA \frac{1}{2 dx}}$$

Substitute the value of [O₂blood](t) obtained from equation (5) shown above, into equation (3), we get

$$\begin{matrix}\frac{{dn}_{{O}_{2} lungs}}{dt} = {O}_{{2}_{inhale}} ( t ) + DA \frac{\frac{[ {O}_{{2}_{{Sp}_{a}}} ] Vblood ( t ) - DA \frac{[ {O}_{2} lungs ] ( t )}{2 dx}}{Vblood - DA \frac{1}{2 dx}}}{dx} - \\ DA \frac{[ {O}_{2} lungs ] ( t )}{dx} \\ = {O}_{{2}_{inhaled}} ( t ) + \frac{DA}{dx} \frac{[ {O}_{{2}_{{Sp}_{a}}} ] Vblood ( t )}{Vblood - DA \frac{1}{2 dx}} - \\ \frac{DA}{dx} [ {O}_{2} lungs ] ( t ) ( 1 + \frac{DA}{Vblood - \frac{DA}{2 dx}} ) \\ = {O}_{{2}_{inhaled}} + \frac{DA}{dx} \frac{[ {O}_{{2}_{{Sp}_{a}}} ] Vblood ( t )}{Vblood - DA \frac{1}{2 dx}} - \\ \frac{DA}{dx} \frac{{n}_{{O}_{2} lungs ( t )}}{Nlungs} ( 1 + \frac{DA}{Vblood - \frac{DA}{2 dx}}\end{matrix} \qquad ( 6 )$$

where Nlungs is the total number of mole of air in the lungs, which is in direct relation to the volume of the lungs and the ambient conditions, which is easily obtained from the breathing/breathing rate sensor. Equation (6) is a first order ordinary differential equation in the form of y′+p(x)y+=Q(x) with solution of the form

$$\frac{{dn}_{{O}_{2} lungs}}{dt} .$$

First, the user wears the three sensors for heartrate, spO2, and breathing rate, while breathing through a gas exchange analyzer. The three sensors provides the values of O₂inhale. [O₂Spa], Vblood, Nlungs, and the gas exchange measures the values of nO₂lungs, and

$$y = \frac{1}{\int ( x )} [ \int I ( x ) Q ( x ) dx + C ] where I ( x ) = {e}^{\int p ( x ) dx} .$$

The missing values are the user specific lung properties D, A, dx, which can be obtained by either statistical methods, e.g., least square error, or by utilizing machine learning and AI for finding the best solution.

Now that we have the user specific parameters that map the lung behavior, we no longer need the gas exchange analyzer, and the user can go freely while wearing the non-intrusive three sensors. Using these three sensors and the user specific lung properties, the model represented in the differential equation above is fully defined, and thus we can solve for the amount of oxygen exchanged.

The amount of oxygen exchange allows us to accurately obtain many important data about the body, one of which is the metabolic rate and calorie/energy expenditure.

An elaboration of the model can be made by adding the effect of the dead space, which is the amount of air in the lungs that does not get replenished and thus the beginning of each inhale, there is always a little amount of air with the lowest concentration of oxygen in it.

An elaboration of the model can be made by including the change of the Nlungs due to the exchange of O2 with CO2, this effect is very small. Another elaboration of the model can be made by include the temperature increase of the inhaled air. Air enters at ambient but leaves closer to the temperature of the body 37C.

Some benefits of such a device:
accurate metabolic rate calculations
accurate BMI calculations
accurate body composition calculations
optimize breathing pattern specific for each user
optimize cardiac operation range based on different types of activities, optimal
spo2-heartrate zones to replace the inaccurate conventional method of heartrate zone only
track activity stress and recovery, and allow for optimizing each for a given fitness goal, and avoid overtraining
track improvement of the cardiac and pulmonary system and body composition.

The various embodiments described above can be combined to provide further embodiments. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
determining a metabolic rate of a user by:
obtaining oxygen consumption of the user;
determining heart rate of the user from a first sensor;
determining oxygen saturation in blood of the user from a second sensor;
determining breath rate of the user from a third sensor;
determining lung boundary properties for the user; and
generating the metabolic rate of the user from the oxygen consumption, heart rate, oxygen saturation, breath rate, and lung boundary properties.

2. The method of claim 1, wherein the first sensor is a heart rate sensor.

3. The method of claim 2, wherein determining the heart rate includes determining a blood flow rate.

4. The method of claim 1, wherein the second sensor is a pulse oximetry sensor.

5. The method of claim 1, wherein the third sensor is a breathing sensor.

6. The method of claim 1, wherein obtaining the oxygen consumption is carried out by a gas exchange analyzer.

7. The method of claim 6, comprising reiterating generating the metabolic rate after obtaining the oxygen consumption.

8. The method of claim 3, wherein the lung boundary properties include a diffusion coefficient, a thickness of a lung membrane, and an area of the lungs.

9. The method of claim 8, comprising obtaining oxygen partial pressure in the blood based on the oxygen saturation, wherein generating the metabolic rate includes using the oxygen partial pressure.

10. The method of claim 1, comprising reiterating determining the heart rate, oxygen saturation, breath rate, or any combination thereof, after generating the metabolic rate.

11. A method comprising:
obtaining a base oxygen consumption of a user from a gas exchange analyzer coupled to a controller;
obtaining, via a plurality of sensors coupled to the controller, base values of the user's blood flow rate, oxygen arterial saturation, and breathing rate;
calculating a metabolic rate of the user based on the oxygen consumption, blood flow rate, oxygen arterial saturation, and breathing rate;
obtaining, via the plurality of sensors, new values of the user's blood flow rate, oxygen arterial saturation, and breathing rate; and
calculating a new metabolic rate based on the new values of the blood flow rate, oxygen arterial saturation, and breathing rate.

12. The method of claim 11, wherein the plurality of sensors includes an oxygen saturation sensor, a lung oxygen concentration sensor, and a heart rate sensor.

13. The method of claim 12, comprising, after calculating the metabolic rate, decoupling the gas exchange analyzer from the controller.

14. The method of claim 11, wherein the monitoring device includes a smartphone, smartwatch, laptop, server, communication device, display device, or processing device.

15. The method of claim 13, wherein the controller communicates with a monitoring device over a network.

16. The method of claim 11, wherein the plurality of sensors includes a pulse oximeter for obtaining the blood flow rate and oxygen arterial saturation.

17. The method of claim 11, comprising calculating, via the controller, an oxygen concentration of blood for the user, based on the blood flow rate and the oxygen arterial saturation.

18. A system comprising:
a controller for calculating a metabolic rate of a user;
a gas exchange analyzer couplable to the controller;
a plurality of sensors coupled to the controller; and
a monitoring device communicating with the controller over a network.

19. The system of claim 18, wherein the plurality of wearable sensors includes an oxygen saturation sensor, a lung oxygen concentration sensor, and a heart rate sensor.

20. The system of claim 18, wherein the controller is a wearable device.
